(19) United States
(12) Patent Application Publication
(10) Pub. No.: US 2006/0251593 A1
(43) Pub. Date: Nov. 9, 2006

(54) COLORED NAIL ENAMEL TREATMENT

(75) Inventors: Alphonse Tribuiani, Naples, FL (US); Peggy Lieber, (US)

Correspondence Address: CHRISTOPHER PARADIES, PH.D., FOWLER WHITE BOGGS BANKER, P.A., 501 E KENNEDY BLVD, STE. 1900, TAMPA, FL, 33602, US

(73) Assignee: Work by Docs, Inc.

(21) Appl. No.: 11/278,919

(22) Filed: Apr. 6, 2006

Related U.S. Application Data
(60) Provisional application No. 60/594,431, filed on Apr. 7, 2005.

(51) Int. Cl. A61K 8/97; A61K 36/534

(52) U.S. Cl. 424/61; 424/747; 424/769

(57) ABSTRACT

Botanicals are combined with colored nail enamel to synergistically improve the topical treatment of nail infections. The combination of two or more of the tea tree oil, Gaultheria procumbens and peppermint with one or more of lactic acid and caprylic acid in a colored nail enamel is believed to improve the efficacy of nail treatment for infected nails or to prevent recurrence of nails with onychomycosis.

### FIELD OF THE INVENTION

The field of the invention is topical antifungal nail treatments.

### BACKGROUND

Topical antifungal nail treatments are known that may be applied to the surface of a nail to inhibit or treat a nail infection, such as a fungal infection or a microbial infection.

Some antimycotic and antimicrobial substances are known that are synthetic or botanical. However, many antimycotic and antimicrobial substances are incompatible with a colored nail enamel, because the appearance of the colored nail enamel is degraded by the activity of the antimycotic or antimicrobial substance. Specifically, it is known that botanical oils cause an uneven appearance of the color of the enamel and may cause degraded surface finish.

Peppermint (Mentha piperita) is a pleasant flavoring agent that has also been used as a botanical medicine. The herb is used as an anti-spasmotic, an antimicrobic and a pleasant after dinner mint that settles the stomach. The active constituent of peppermint, which is found in the leaves and flowering tops of the peppermint plant, is menthol. Peppermint oil is an extract from the peppermint plant which may contain anywhere from 50% to nearly 85% of menthol.

Tea tree oil (Melaleuca alternifolia) is a natural antimycotic. The term Antimycotic means a substance that is capable of inhibiting the growth of fungi. An antimycotic agent destroys or prevents the growth of fungi. Use of tea tree oil as an antimycotic is described in U.S. Pat. No. 6,740,326; however, this reference does not disclose its use in a colored enamel. Instead, one of the claims teaches away from the use of tea tree oil in a nail enamel.

Gaultheria procumbens, which is commonly known as wintergreen, may have some antimicrobial or antimycotic properties.

U.S. Pat. No. 5,968,986 discloses the use of tea tree oil and also discloses the use of pigments; however, the use of tea tree oil and pigments in combination with each other in a nail enamel is not disclosed.

Treatment of nail infections can require prolonged medical therapy, which may be invasive, uncomfortable and even toxic. Cure rates and patient tolerance of such medical therapies are less than satisfactory.

U.S. Pat. No. 6,013,677 discloses that onychomycosis is just one example of myriad of microbial etiologies of nail disfigurement that must be treated. This references discloses a need for a substance that can treat polymicrobial infections that are spawned by multiple infectious agents, such as bacteria, molds, spores, viruses and fungi.

U.S. Pat. No. 4,967,730 discloses an antimycotic nail varnish, which contains a 1-hydroxypyridone as a antimycotic agent. However, 1-hydroxypyridone has limited application, because it is effective only against mycotic organisms and not bacteria.

U.S. Pat. No. 5,487,776 discloses an antifungal nail lacquer that contains griseofulvin as an antifungal agent. Griseofulvin is only an antifungal agent and is not antibacterial.

Colors and pigments that are useful in a nail enamel are known, such as insoluble and colored polymeric materials, opacity regulating agents such as titanium dioxide, aluminum silicate, tinting agents such as iron oxide, and pearlescent agents such a guanine bismuth oxide chloride and pigments such as D&C Red 6, D&C Red 30, D&C Red 36, D&C Red 9, D&C Red 7, FDC Yellow 5 and FDC Yellow 6. The amount of pigment used in a nail enamel composition can vary depending upon the type of pigment chosen, but generally does not exceed about 15 weight percent of the nail enamel.

U.S. Pat. No. 6,013,677 discloses some ranges for pigments and coloring agents, which are incorporated herein by reference.

U.S. Pat. No. 6,413,555 discloses a natural nail treatment using between about 10 and about 17 ml of tea tree oil, between about 10 and 17 ml of lavender oil and between about 1 and 3.5 ml of eucalyptus oil in a total of 30 ml of liquid. The reference discloses that between 3% and 5% of the population in the U.S. is affected by nail fungus and treatment options available are unsatisfactory. Such nail infections reside under the nail in an area that is difficult to access by conventional topical treatments, and systemic treatments administered orally, have undesirable side effects. The reference discloses that treatment of onychomycosis using natural skin care products based on herbs and natural oils is much more cost effective than the use of pharmaceuticals. While U.S. Pat. No. 6,413,555 discloses a natural nail treatment, the natural nail treatment disclosed is not suitable as a colored nail enamel.

U.S. Pat. No. 6,703,003 discloses a cyanoacrylate composition containing one of a list of oils, including tea tree oil and preferably avocado and jojoba oil to suppress heat generation upon hardening of the cyanoacrylate and to prevent keratinization of the nail. JP-A-2-91012 discloses use of cyanoacrylates as enamel coating composition for nails.

U.S. Pat. No. 6,821,508 discloses research efforts focusing on improving penetration of a keratin matrix by chemically modifying the keratin matrix, citing Kobayashi et al. in Chem Pharm Bull 46 (11):1797-1802 (1998). Examples of compounds which interact with keratin are mercaptan compounds, sulfites and bisulfides, keratolytic agents, and surfactants. Examples of these are listed in U.S. Pat. No. 6,821,508 and are incorporated herein by reference as background. Other beneficial agents are likewise disclosed, such as antipsoriatic drugs, nail growth promoters and nail conditioners.

U.S. Pat. No. 6,264,934 discloses vinylic copolymers derived from one or more ethylenically unsaturated monomers containing an acid, one or more ethylenically unsaturated monomers containing modifying groups that did not substantially interfere with the solubility in non-water miscible organic solvents, and one or more ethylenically unsaturated organosiloxane chains. Specifically, a nail polish is disclosed that mixes the following ingredients at room temperature in a ball mill overnight: 16 wt. % of solids polymer, 2 wt. % of red iron oxide, 48 wt. % of butyl acetate, 24 wt. % of ethyl acetate, 8 wt. % isopropanol.

### SUMMARY OF THE INVENTION

A combination of natural substances is added to a colored enamel for treating or preventing nail infections. The natural substances are botanicals that are mixed together and mixed with the colored enamel. The colored enamel serves to hide the disfigurement of onychomycosis, while not interfering with or actually improving the efficacy of the combination of antimicrobial botanicals. The antimicrobial botanicals are selected to provide antimycotic and antibacterial properties to the colored nail enamel, without degrading the appearance of the colored nail enamel.

One object of the present invention is to incorporate inexpensive botanicals in a colored nail enamel, which helps to hide the disfigurement caused by infections, such as onychomycosis, while treating the infection or inhibiting reinfection.

### DETAILED DESCRIPTION OF PREFERRED EMBODIMENTS OF THE INVENTION

This detailed description provides specific examples of the invention, but the invention should not be limited merely to the examples disclosed. Instead, the invention should be limited only by the claims that may eventually issue. Many variations in the treatment system, changes in specific components of the treatment system and uses of the treatment system will be readily apparent to those familiar with the field of treating nail infections after reading the disclosure.

In one example, an effective amount of tea tree oil is combined with an effective amount of Gaultheria procumbens, lactic acid, and caprylic acid to form an antimicrobial composition, which can be mixed with a nail enamel, such as polymers of an acrylate, acrylic co-polymers, cyanoacrylates, methacrylic acid and esters of methacrylic acid, cellulose, nitrocellulose, cellulose acetates, nylon, polyvinyl acetates, formaldehyde resins, and combinations of these, without substantially degrading the appearance of the nail polish. The antimicrobial composition is mixed with a nail enamel in a proportion such that the appearance of the nail enamel is not degraded by the addition to the antimicrobial composition, but still provides an effective amount of antimicrobial to treat nail infections. For example, the antimicrobial composition may comprise less than 25 wt. % of the mixture of nail enamel and antimicrobial composition. The nail enamel may also comprise a colorant, a pigment and/or a pearlescent, in an amount effective to obscure the disfigurement caused by onychomycosis.

In one example, an antifungal nail system provides a clear nail enamel having a higher concentration of active ingredients and a colored nail enamel having a lower concentration of active ingredients, which nevertheless provides an effective amount of active ingredients for temporary cosmetic use during treatment of the nails. This overcomes one of the problems of previous nail treatments, which often fail when a patient fails to use the nail treatment for cosmetic reasons. The anti-fungal, colored nail enamel is prescribed for short periods of use, when the patient would otherwise fail to use the clear antifungal nail enamel, providing for continuous treatment of the infected nails and preventing reinfection of successfully treated nails during the period of recovery.

In another example, the antimicrobial composition may also comprise peppermint. For example, equal parts of tea tree oil, Gaultheria procumbens and peppermint may be mixed with either lactic acid or caprylic acid or both.

It is believed that the lactic acid, caprylic acid or both the lactic acid and caprylic acid assist in penetration of the tea tree oil, Gaultheria procumbens and peppermint into and underneath the nail surface. Thus, the addition of lactic and/or caprylic acids greatly increase the effectiveness of the other botanicals, which allows less of the botanicals to be mixed with the colored enamel.

It is also believed that the combination of tea tree oil, Gaultheria procumbens, and peppermint provide treatment of polymicrobial infections. It is believed the combination of two or more of tea tree oil, Gaultheria procumbens and peppermint with one or more or lactic acid and caprylic acid provides a synergistic effect that is otherwise not realized. Thus, the combination of botanicals provides an effective treatment for onychomycosis in concentrations within a nail enamel that do not degrade the appearance of the nail enamel itself.

Furthermore, it is believed, without being limiting, that the incorporation into a nail enamel also synergistically improves the treatment efficacy of the botanicals. Specifically, the nail enamel allows the botanicals to fuse into the nail surface, while providing a durable surface that resists wear and accidental removal during an extended period. In addition, treatment effectiveness is further improved by making use of a nail treatment more desirable to the patient, providing a treatment that hides the disfigurement associated with nail infections.

In one embodiment, equal amounts of tea tree oil, Gaultheria procumbens, peppermint, lactic acid and caprylic acid are combined in a nail enamel, such as a colored nail enamel, in a concentration between 3 and 10 wt. % of the combined ingredients.

What is claimed is:

1. A nail infection treatment system for treating fungal infections in a nail enamel comprising:
an effective amount of an antifungal selected from the group of antifungals consisting of tea tree oil, Gaultheria procumbens, and peppermint and combinations thereof; and an effective amount of at least one substance selected from the group consisting of lactic acid and caprylic acid and combinations thereof; and a colored nail enamel, wherein the colored nail enamel is mixed together with the effective amount of an antifungal and the effective amount of the at least one substance.

2. The nail infection treatment system of claim 1, wherein the nail enamel comprises at least one component selected from the group consisting of colorant, pigment, and a pearlescent or combinations thereof.

3. The nail infection treatment system of claim 1, wherein the treatment system is mixed with the nail enamel in a proportion such that the appearance of the nail enamel is not degraded by the treatment system but provides an effective amount of nail infection treatment.

4. The nail infection treatment system of claim 1, wherein the effective amount of the antifungal includes tea tree oil, and Gaultheria procumbens, and the effective amount of at least one substance includes lactic acid and caprylic acid.

5. The nail infection treatment system of claim 1, wherein the nail enamel is selected from the group consisting of acrylate, acrylic co-polymers, cyanoacrylates, methacrylic acid and esters of methacrylic acid, cellulose, nitrocellulose, cellulose acetates, nylon, polyvinyl acetates, formaldehyde resins, and combinations thereof.

6. The nail infection treatment system of claim 1, wherein the treatment system includes effective equal amounts of tea tree oil, Gaultheria procumbens, lactic acid and caprylic acid, that are mixed with the nail enamel.

7. The nail infection treatment system of claim 1, wherein the treatment system comprises equal effective amounts of tea tree oil, Gaultheria procumbens, and peppermint in combination with an effective amount of at least one substance selected from the group consisting of lactic acid and caprylic acid, and combinations thereof.

8. The nail infection treatment system of claim 1, where the system is used for treating fungal infections.

9. The nail infection treatment system of claim 8, wherein the fungal infection includes onychomycosis.

10. A method of treating fungal infection of a nail enamel, comprising:
selecting a combination of an effective amount of at least two substances selected from the group consisting of tea tree oil, Gaultheria procumbens, and peppermint and combinations thereof;
mixing the combination with an effective amount of at least one substance selected from the group consisting of lactic acid and caprylic acid and combinations thereof;
compounding the combined substances into a compound; and
administering the compound to a patient for nail infections by mixing the compound with the nail enamel.

11. The method of claim 10, wherein the selecting step selects a combination of an effective amount of the tea tree oil with an effective amount of the Gaultheria procumbens, the lactic acid and the caprylic acid, to form a compound for treating nail infections.

12. The method of claim 10, wherein the selecting step selects a combination of an effective amounts of the tea tree oil, the Gaultheria procumbens, the lactic acid and the caprylic acid.

13. The method of claim 10, wherein the selecting step selects a combination of equal effective amounts of the tea tree oil, the Gaultheria procumbens, and the peppermint in combination with an effective amount of at least one substance selected from the group consisting of the lactic acid and the caprylic acid, and combinations thereof, to form the compound for treating nail infections.

14. The method of claim 10, wherein the selecting step selects a combination comprising: equal effective amounts of the tea tree oil, the Gaultheria procumbens, the peppermint, the lactic acid and the caprylic acid.

15. The method of claim 10, wherein the selecting step selects a combination of equal effective amounts of the tea tree oil, the Gaultheria procumbens, and the peppermint in combination with an effective amount of at least one substance selected from the group consisting of the lactic acid and the caprylic acid.

16. A composition for treating nail infections comprising: a combination of an effective amount of at least two antifungal substances selected from an effective amount of the group consisting of tea tree oil, Gaultheria procumbens, and peppermint and combinations thereof, and an effective amount of at least one substance selected from the group consisting of lactic acid and caprylic acid and combinations thereof.

17. The composition of claim 16, wherein the effective amount of the tea tree oil is combined with an effective amount of the Gaultheria procumbens, the lactic acid and the caprylic acid.

18. The composition of claim 16, wherein the combination includes equal amounts of the tea tree oil, the Gaultheria procumbens, the lactic acid and the caprylic acid.

19. The composition of claim 16, wherein the combination includes equal effective amounts of the tea tree oil, the Gaultheria procumbens, and the peppermint in combination with an effective amount of at least one compound selected from the group consisting of the lactic acid and the caprylic acid, and combinations thereof.

20. The composition of claim 16, wherein the combination comprises equal effective amounts of the tea tree oil, the Gaultheria procumbens, the peppermint, the lactic acid and the caprylic acid.
